(19) United States
(12) Patent Application Publication
(10) Pub. No.: US 2002/0087066 A1
(43) Pub. Date: Jul. 4, 2002

(54) Method for generating magnetic resonance images

(76) Inventor: Marion Hellinger, Uttenreuth (DE)

Correspondence Address: SCHIFF HARDIN & WAITE, 6600 SEARS TOWER, 233 S WACKER DR, CHICAGO, IL, 60606-6473, US

(21) Appl. No.: 10/004,466

(22) Filed: Nov. 1, 2001

(30) Foreign Application Priority Data
Nov. 9, 2000 (DE) 10055417.2

(51) Int. Cl. A61B 5/055

(52) U.S. Cl. 600/410

(57) ABSTRACT

A method and associated apparatus for generating magnetic resonance images of a subject with a magnetic resonance apparatus on the basis of a sequence in the framework of an examination of the subject contains the following steps: the magnetic resonance apparatus is supplied with subject-specific and/or examination-specific parameters, and the magnetic resonance apparatus determines optimum settings and/or setting ranges of sequence parameters for a combination of the supplied subject-specific and/or examination-specific parameters for generating magnetic resonance images.

### BACKGROUND OF THE INVENTION

1. Field of the Invention

The invention is directed to a method for generating magnetic resonance images of a subject with a magnetic resonance apparatus based on a subject examination sequence.

2. Description of the Related Art

Magnetic resonance technology is a known technique for acquiring images of the inside of the body of a subject to be examined. In a magnetic resonance apparatus, rapidly switched gradient fields that are generated by a gradient system are superimposed on a static basic magnetic field that is generated by a basic field magnet. The magnetic resonance apparatus also comprises a radio frequency system that, for triggering magnetic resonance signals, beams radio frequency signals into the subject and picks up the magnetic resonance signals that are generated; magnetic resonance images are generated based on this. In one embodiment, the radio frequency system comprises a stationary antenna that, for example, is fashioned as a “whole-body antenna” that can be utilized as a transmission and as a reception antenna. In addition, “local antennas” are utilized for improving a signal-to-noise ratio. These are antennas that are adapted to a size of a region of the subject to be imaged. For example, there are local antennas for, e.g., examination of a spinal column or of a female breast that are permanently installed in a bearing mechanism of the apparatus or that can be latched at fixed positions on the bearing mechanism. On the other hand, there are also freely movable local antennas, for example, flexible antennas and extremity antennas. In one embodiment, the local antenna can be fashioned as a pure reception antenna, in which, for example, the stationary antenna is then utilized as a transmission antenna.

By positioning the region to be imaged in an imaging volume of the apparatus, the reception antenna is attenuated depending on the length and mass of the subject as well as on the type of region to be imaged. In order to achieve a good image quality, the previously mentioned attenuation is compensated in one embodiment in that a matching device of the reception antenna is correspondingly set in a procedure preparatory to the image pick-up.

For improving the image quality given local reception antennas, a compensation within magnetic resonance images is implemented with a normalization filter. To this end, the normalization filter reduces signal intensities from antenna-proximate regions of the region to be imaged and instead boosts signal intensities from antenna-remote regions of the region to be imaged. Particularly given employment of modern sequence techniques, the apparatus automatically determines a characteristic of the reception antenna needed for the image normalization. In a magnetic resonance examination of a patient, the apparatus is informed of a mass and an age or date of birth of the patient for limiting an applied radio frequency power to a patient-specific limit (specific absorption rate) that is harmless for the patient.

For generating magnetic resonance images of the region to be imaged, the magnetic resonance apparatus is controlled based on a sequence prescribable by the operator of the apparatus; the operator can prescribe a sequence type, for example, a spin echo sequence, a gradient echo sequence, a multi-echo sequence, a sequence with preparation of the magnetization, an echo planar sequence, etc. The operator can set sequence parameters for the selected sequence type, for example a repetition time, an echo time, a matrix size of the magnetic resonance image, a field of view, a thickness of a slice to be registered, a flip angle, etc.

A majority of magnetic resonance examinations are a matter of routine examinations, for example, of the cervical spine, knee or head of a patient, for which a reproducibly high magnetic resonance image quality is desired independently of the patient to be examined. The image quality that is achieved is dependent on the experience of the respective operator. An inexperienced operator, for example, will examine each patient with identically selected sequence parameters, which leads to totally different image qualities dependent on the patient. A similar effect arises because the operators (for example, the physician and medical technicians) at magnetic resonance apparatus change frequently, so that the individual operators are often only inadequately familiarized with the equipment, procedures, and patients.

### SUMMARY OF THE INVENTION

It is therefore an object of the invention to provide a method for achieving a reproducibly high magnetic resonance image quality.

This object is inventively achieved by a method for generating magnetic resonance images of a subject with a magnetic resonance apparatus based on a sequence in a framework of a subject examination comprising: providing the magnetic resonance apparatus with specific parameters that comprise at least one of subject-specific and examination-specific parameters; and determining, by the magnetic resonance apparatus, setting parameters that comprise at least one of optimum settings and setting ranges of sequence parameters, for a combination of the specific parameters for generating magnetic resonance images.

The method may further comprise the step of allocating the setting parameters to the specific parameters via a table linkage. A neural network may be used for determining setting parameters for the specific parameters.

The subject-specific parameters may include mass, height, sex, age, date of birth, stature, body measurement, fat part, and proton density. The examination-specific parameters may include sequence type, contrast preselection, and region of the subject to be imaged. The setting parameters may include field of view, repetition time, echo time, matrix size, thickness of a slice to be imaged, number of averagings, bandwidth, and cut-off of a normalization filter.

The specific parameters may be entered by an operator of the magnetic resonance apparatus at the magnetic resonance apparatus, but specific parameters may also be automatically determined.

The objects of the invention may also be achieved by an apparatus for generating magnetic resonance images of a subject with a magnetic resonance apparatus based on a sequence in the framework of a subject examination comprising: a field magnetic system for producing magnetic resonance images; an operating mechanism and a display for entering information; and a central control system that connects the operating mechanism and the field magnetic system, the central control system comprising a processor, a database, and algorithms for controlling the apparatus for generating magnetic resonance images based on a sequence in a framework of a subject examination; in which the database comprises structures with specific parameters that comprise at least one of subject-specific and examination-specific parameters, the database further comprising structures with setting parameters that comprise at least one of optimum settings and setting ranges of sequence parameters, for a combination of the specific parameters for generating magnetic resonance images. The specific parameters and setting parameters may include values as described above.

As a result of the inventive method, a reproducibly high quality is achieved for all magnetic resonance images independently of the wide variations in subjects to be examined and independently of the experiences of a respective operator of the magnetic resonance apparatus.

### DESCRIPTION OF THE DRAWINGS

Further advantages, features and details of the invention derives from the exemplary embodiments described below on the basis of the drawings.

FIG. 1 is a pictorial block diagram of a magnetic resonance apparatus; and

FIG. 2 a flow diagram for a method for determining optimum settings and/or setting ranges of sequence parameters.

### DETAILED DESCRIPTION OF THE INVENTION

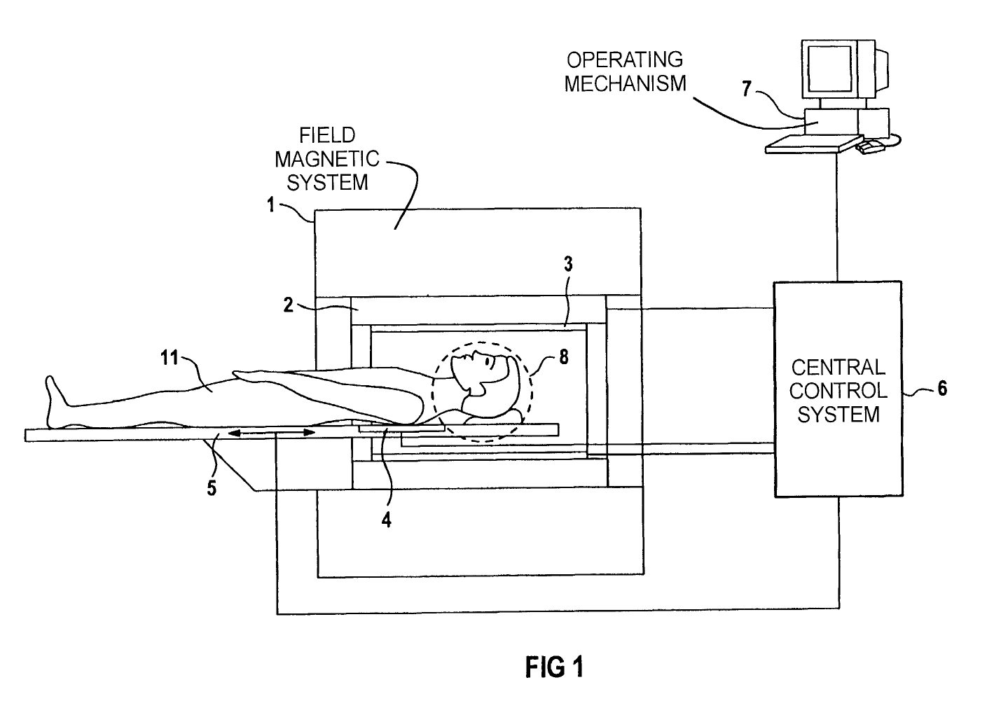

FIG. 1 diagrams a magnetic resonance apparatus that comprises a basic field magnet system 1 for generating a basic magnetic field and a gradient coil system 2 for generating gradient fields. For applying radio frequency signals, the apparatus comprises a stationary antenna 3 as well as a local antenna 4 for picking up the magnetic resonance signals that are generated. The gradient coil system 2 is connected to a central control system 6 for controlling currents in the gradient coil system 2 based on a selected sequence. The antennas 3 and 4 are likewise connected to the central control system 6 for controlling the radio frequency signals to be emitted according to the selected sequence as well as for the further processing and storing of the magnetic resonance signals picked up by the antennas 3 or 4. Furthermore, the apparatus comprises a movable bearing device 5 on which a patient 11 to be examined is placed. The local antenna 4 is fashioned to be latchable into the movable bearing device 5. For controlling a displacement of the movable bearing device 5, for example, in order to position a region of the patient 11 to be examined in an imaging volume 8 of the apparatus, the bearing device 5 is also correspondingly connected to the central control system 6. The central control system 6 is connected to a display and operating mechanism 7 via which operator inputs, for example, the desired sequence type and sequence parameters, are supplied to the central control system 6. Among other things, the generated magnetic resonance images are displayed at the display and operating mechanism 7.

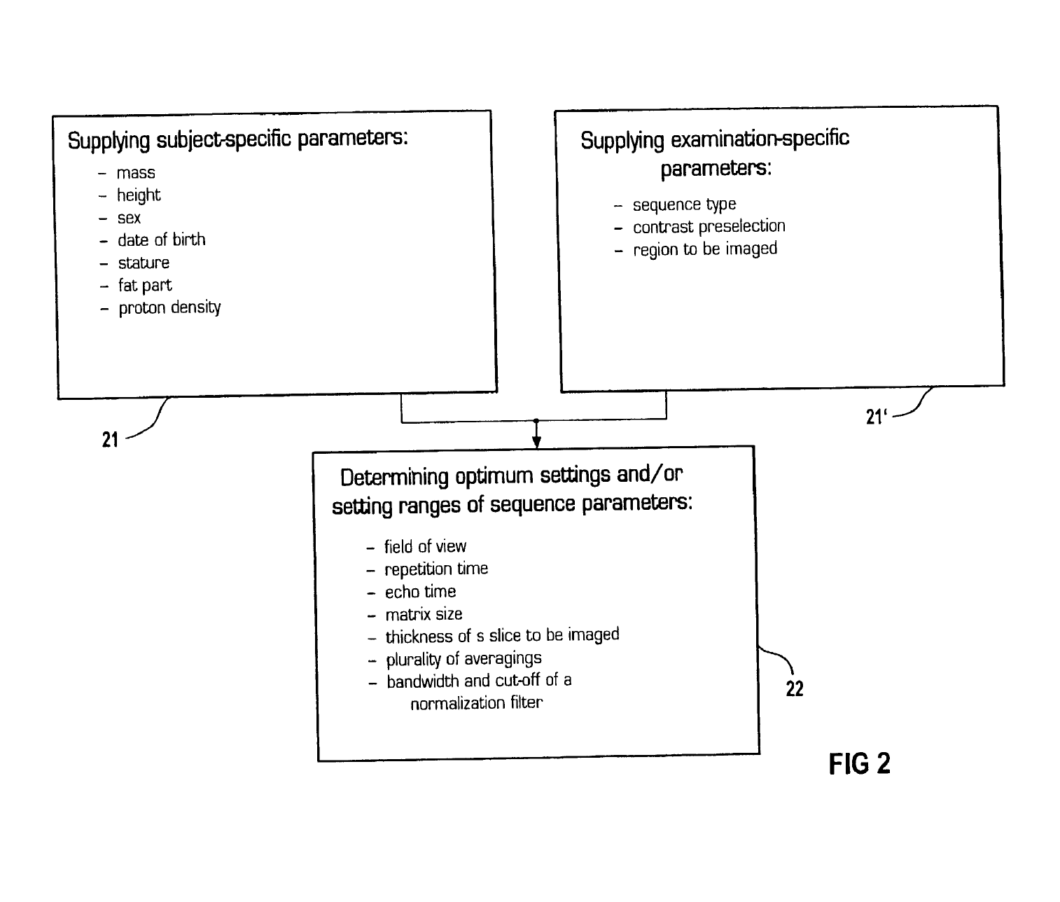

FIG. 2 is a flow diagram for a method for determining optimum settings and/or setting ranges for sequence parameters. The magnetic resonance apparatus shown in FIG. 1 is accessed by way of the following example. In the first two steps 21 and 21′ of the flowchart, subject-specific and/or examination-specific parameters are input at the magnetic resonance apparatus by the operator, potentially with the assistance of further measuring instruments, and/or are automatically determined by the magnetic resonance apparatus following a start signal that, e.g., is triggered by the operator.

A mass and an age or a date of birth of the patient 11, e.g., may be communicated to the apparatus—for example, by the operator—in every magnetic resonance examination of a patient for limiting an applied radio frequency power to a patient-specific limit value that is harmless for the patient. In another embodiment, the magnetic resonance apparatus takes these subject specific parameters from a patient data bank correspondingly coupled to the central control system 6. The subject-specific parameters of height, sex and stature (or other body measurement, including physical measurement dimensions, tissue characteristics, etc.) can also be handled in a way similar to the subject-specific parameters of age and mass.

Depending on the diagnostic requirements, the operator may set a sequence type, for example, a spin echo sequence, a gradient echo sequence, a multi-echo sequence, a sequence with preparation of the magnetization, an echo planar sequence, etc., at the operating and display device 7 in step 21′ of the flowchart of FIG. 2 in combination with a contrast pre-selection in view of a T1 or T2 weighting.

At the beginning of a magnetic resonance examination, the patient 11 is positioned on the bearing device 5 that is withdrawn as far as possible from the basic field magnet system 1. The patient 11 is positioned according to the region to be imaged, this having already been determined at the start of the examination. When, for example, the cervical spine of the patient 11 is the region to be imaged, then the patient 11 is positioned head-first on the bearing mechanism 5. Whether the patient 11 is positioned on her back or on his stomach can also be selected dependent on the diagnostic requirements. The operator correspondingly inputs the region to be imaged and/or the bearing mode of the patient 11 at the display and control device 7 as examination-specific parameters. As a supplement to or replacement for these examination-specific parameters, an organ of the patient 11 under examination can also be indicated.

For the actual implementation of the examination, the bearing mechanism 5 is displaced such that the region to be imaged is positioned in the imaging volume 8 of the apparatus. Following this, for example, an overview image may be registered given the parameters that have already been determined and input into consideration.

Before a registration of anatomical and/or functional magnetic resonance images, optimum settings and or setting ranges of sequence parameters are determined in a step 22 of the flowchart from the subject-specific and/or examination-specific parameters in view of a good magnetic resonance image quality and are offered to the operator at the display and operating device 7 for acceptance, selection, modification and rejection. The following are important sequence parameters for the image quality: a field of view, a repetition time, an echo time, a matrix size, a plurality of averagings, a thickness of a slice to be imaged and a cut-off and/or a bandwidth of a normalization filter. When, for example, a heavyweight patient is present and when the local antenna 4 is utilized as a reception antenna, then a setting with low values for the bandwidth and the cut-off of the filter are recommended to the operator in view of the normalization filter.

The optimum settings and/or setting ranges of the sequence parameters following modified frame conditions (for example, following an administration of a contrast agent) may be adapted to the modified conditions and offered anew. In one embodiment, an optimum amount of contrast agent may also be proposed.

In one embodiment, the optimum settings and/or setting ranges of the sequence parameters may be allocated to a combination of subject-specific and/or examination-specific parameters via a table linkage. A table on which this linkage is based is generated, for example, in the framework of a series of examinations and/or on the basis of empirical values and is stored in the central control system 6. The table can be expanded with every new examination.

The allocation on the basis of a table is explained below by way of example for a T2-weighted spinal column examination of a patient of normal weight and an overweight patient. The patient of normal weight is identified as such based on subject-specific parameters (e.g., height equal to 1.6 m and mass equal to 60 kg) input by the operator. The operator inputs age and/or date of birth, sex as well as the type of bearing of the normal-weight patient on the bearing mechanism 5 head-first and lying on his/her back. The optimum settings and/or setting ranges for the sequence parameters for this combination of subject-specific and examination-specific parameters are deposited in the table stored in the central control system 6, these being offered to the operator at the display and operating device 7. For example, thirteen might be provided as a number of slices to be imaged and two might be provided as a number of averagings. In contrast to the normal-weight patient, the heavyweight patient parameters (e.g., 1.8 m as height and 100 kg as mass) may be input for the heavyweight patient. Due to an anticipated, higher fat signal and a greater length of the heavyweight patient compared to the normal-weight patient, the number plurality of slices to be imaged might be fifteen, and the number of averagings might be three, with higher numbers as sequence parameters compared to the normal-weight patient, being provided according to the tabular linkage.

In another embodiment, a neural network implemented in the central control system 6 may determine the optimum settings and/or setting ranges of the sequence parameters for combinations of subject-specific and/or examination-specific parameters. To that end, the neural network is supplied with an adequately great plurality of combinations for achieving an adequately long training phase in combination with a statement as to whether a good or poor image quality can thus be achieved. The neural network can continue to learn with every new examination.

In one embodiment, the operator may be offered a help function given a modification of sequence parameters which points out the optimum settings and/or setting ranges for a high image quality. When, for example, a T1-weighted spin echo sequence is preselected and the operator raises the repetition time such that the T1 contrast behaviour is lost, then the help function appropriately points this out to the operator.

In another embodiment, movements on the part of the patient are at least pointed out after a definition of the sequence parameters and a beginning of the actual image generation, since these can likewise lead to deteriorations of the image quality.

The above-described methods and apparatus are illustrative of the principles of the present invention. Numerous modifications and adaptations will be readily apparent to those skilled in this art without departing from the spirit and scope of the present invention.

What is claimed is:

1. A method for generating magnetic resonance images of a subject with a magnetic resonance apparatus based on a sequence in a framework of a subject examination comprising:
providing said magnetic resonance apparatus with specific parameters that comprise at least one of subject-specific and examination-specific parameters; and
determining, by said magnetic resonance apparatus, setting parameters that comprise at least one of optimum settings and setting ranges of sequence parameters, for a combination of said specific parameters for generating magnetic resonance images.

2. The method according to claim 1, further comprising:
allocating said setting parameters to said specific parameters via a table linkage.

3. The method according to claim 1, wherein said determining setting parameters is performed with a neural network for said specific parameters.

4. The method according to claim 1, wherein at least one of said subject-specific parameters is selected from the group consisting of mass, height, sex, age, date of birth, stature, body measurement, fat part, and proton density.

5. The method according to claim 1, wherein at least one of said examination-specific parameters is selected from the group consisting of sequence type, contrast preselection, and region of the subject to be imaged.

6. The method according to claim 1, further comprising:
entering at least one of said specific parameters by an operator of said magnetic resonance apparatus at said magnetic resonance apparatus.

7. The method according to claim 1, further comprising:
automatically determining at least one said specific parameters by said magnetic resonance apparatus.

8. The method according to claim I wherein at least one of said setting parameters are selected from the group consisting of field of view, repetition time, echo time, matrix size, thickness of a slice to be imaged, number of averagings, bandwidth, and cut-off of a normalization filter.

9. An apparatus for generating magnetic resonance images of a subject with a magnetic resonance apparatus based on a sequence in the framework of a subject examination comprising:
a field magnetic system for producing magnetic resonance images;
an operating mechanism and a display for entering information; and
a central control system that connects said operating mechanism and said field magnetic system, said central control system comprising a processor, a database, and algorithms for controlling said apparatus for generating magnetic resonance images based on a sequence in a framework of a subject examination;
wherein,
said database comprises structures with specific parameters that comprise at least one of subject-specific and examination-specific parameters, said database further comprising structures with setting parameters that comprise at least one of optimum settings and setting ranges of sequence parameters, for a combination of said specific parameters for generating magnetic resonance images.

10. The apparatus according to claim 9, wherein said subject-specific parameters comprise at least one entity selected from the group consisting of mass, height, sex, age, date of birth, stature, body measurement, fat part, and proton density.

11. The apparatus according to claim 9, wherein said examination-specific parameters comprise at least one entity selected from the group consisting of sequence type, contrast preselection, and region of the subject to be imaged.

12. The apparatus according to claim 9, wherein said setting parameters comprise at least one entity selected from the group consisting of field of view, repetition time, echo time, matrix size, thickness of a slice to be imaged, number of averagings, bandwidth, and cut-off of a normalization filter.
